(19) United States
(12) Patent Application Publication
(10) Pub. No.: US 2026/0000372 A1
(43) Pub. Date: Jan. 1, 2026

(54) DETECTION SUBSTRATE, X-RAY IMAGING SYSTEM, AND IMAGE CORRECTION METHOD AND APPARATUS

(71) Applicants: Beijing BOE Optoelectronics Technology Co., Ltd., Beijing (CN); BOE Technology Group Co., Ltd., Beijing (CN)

(72) Inventors: Chuncheng CHE, Beijing (CN); Shuai XU, Beijing (CN); Xin LI, Beijing (CN); Xuecheng HOU, Beijing (CN); Guan ZHANG, Beijing (CN); Zhenqian ZHAO, Beijing (CN); Chao CHEN, Beijing (CN)

(21) Appl. No.: 18/880,581

(22) PCT Filed: Apr. 22, 2024
(86) PCT No.: PCT/CN2024/089120

§ 371 Date: Jan. 2, 2025

(30) Foreign Application Priority Data
May 22, 2023 (CN) 202310582809.9

(51) Int. Cl. A61B 6/42; A61B 6/00; G06T 5/50

(52) U.S. Cl. CPC A61B 6/4233; A61B 6/5258; A61B 6/542; G06T 5/50; G06T 2207/10116; G06T 2207/20224

(57) ABSTRACT

The detection substrate comprises an automatic exposure detection unit, a control unit, a gate driving unit, and a data reading unit, wherein the automatic exposure detection unit is configured to send a first notification signal to the control unit when detecting that exposure is started; the control unit is configured to output, in a scanning time period, a first signal and a second signal to the gate driving unit, output the first signal to the end of the current frame when the first notification signal is received; the gate driving unit is configured to control the starting sequence of a plurality of detection units according to the first signal and the second signal in the scanning time period; and the data reading unit is configured to read data of a data line according to a data reading signal in the scanning time period.

### CROSS-REFERENCE TO RELATED APPLICATIONS

The present application is a U.S. National Phase Entry of International Application No. PCT/CN2024/089120 having an international filing date of Apr. 22, 2024, which claims priority to the Chinese Patent Application NO. 202310582809.9, filed to the CNIPA on May 22, 2023 and entitled “Detection Substrate, X-ray Imaging System, and Image Correction Method and Apparatus”. The above-identified applications are incorporated into the present application by reference in their entireties.

### TECHNICAL FIELD

Embodiments of the present disclosure relate to, but are not limited to, the technical field of X-ray imaging, and particularly relate to a detection substrate, an X-ray imaging system, and an image correction method and apparatus.

### BACKGROUND

In recent years, X-ray inspection has been widely used in various fields such as medical treatment, safety, non-destructive testing and scientific research. At present, a common X-ray detection technology is the X-ray Digital Radiography (DR) detection technology that appeared in the late 1990s. The X-ray digital photography detection technology uses Flat Panel Detector (FPD), whose pixel size can be less than 0.1 mm, and whose imaging quality and resolution are almost comparable to those of film photography system. The X-ray digital photography detection technology also overcomes the shortcomings in film photography system, and provides convenience for the computer to process images.

### SUMMARY

The following is a summary of subject matters described herein in detail. This summary is not intended to limit the protection scope of claims.

An embodiment of the present disclosure provides a detection substrate including an automatic exposure detection unit, a control unit, a gate driving unit, and a data reading unit, wherein the detection substrate further includes a plurality of gate lines and a plurality of data lines arranged crosswise and a plurality of detection units defined between the gate lines and the data lines, the plurality of detection units being arranged in an array, wherein:

the automatic exposure detection unit is configured to transmit a first notification signal to the control unit when detecting the start of exposure;

the control unit is configured to: periodically output a first signal and a second signal to the gate driving unit and the data reading unit during a scanning time period; when the first notification signal is received during the scanning time period, output the first signal until the end of the current frame, and interrupt the output of the second signal; acquire data read by the data reading unit during a first scanning time period after the exposure is completed, and generate an initial image according to the acquired data;

the gate driving unit is configured to control an activation sequence of the plurality of detection units according to the first signal and the second signal during the scanning time period, the first signal being used to determine the scanning time of a row in a frame, and the second signal being used to determine position of the row of detection units which are turned on;

the data reading unit is configured to read data of the plurality of data lines during the scanning time period.

An embodiment of the present disclosure also provides an X-ray imaging system comprising an X-ray generator and a detection substrate, wherein:

the X-ray generator is configured to emit X-rays, and stop emitting X-rays according to the received time for current X-ray exposure;

the control unit is further configured to detect a position of an interrupted row when the output of the second signal is interrupted, calculate time for the current X-ray exposure according to the detected position of the interrupted row, wherein the time for the current X-ray exposure=T−ΔT+W, with T being a scanning time of a row in a frame, ΔT being a scanning time of a row before the current frame is interrupted, and W being a preset exposure window width, and send the calculated time for the current X-ray exposure to the X-ray generator so that the X-ray generator stops emitting X-rays according to the time for the current X-ray exposure.

An embodiment of the present disclosure also provides an image correction method for a detection substrate, the method comprising:

obtaining an initial image acquired by the detection substrate, a position of the current interrupted row, and a compensation value, wherein the position of the interrupted row is a position of the row being scanned when start of exposure is detected and the output of the second signal is interrupted;

using the compensation value to compensate for the gray scale of pixels before or after the interrupted row in the initial image to obtain a corrected image.

An embodiment of the present disclosure further provides an image correction apparatus for a detection substrate, the image correction apparatus includes a memory and a processor coupled to the memory for storing instructions, the processor is configured to perform the steps of the image correction method for the detection substrate described in any embodiment of the present disclosure.

An embodiment of the present disclosure further provides a computer-readable storage medium storing a computer program thereon, the program, when executed by the processor, implements the image correction method for the detection substrate described in any embodiment of the present disclosure.

Other aspects may be comprehended upon reading and understanding drawings and detailed description.

### BRIEF DESCRIPTION OF DRAWINGS

Accompanying drawings are used for providing further understanding of technical solutions of the present disclosure, constitute a portion of the specification, and are used for explaining the technical solutions of the present disclosure together with embodiments of the present disclosure, but do not constitute limitations on the technical solutions of the present disclosure. Shapes and sizes of various components in the drawings do not reflect actual scales, but are only intended to schematically illustrate contents of the present disclosure.

FIG. 1 is a schematic diagram of a driving timing of an X-ray imaging system in an automatic exposure detection mode;

FIG. 2 is a schematic diagram of gray scale differences in images acquired when a detection substrate receives an exposure request signal at different positions;

FIG. 3A is a schematic diagram of the structure of a detection substrate according to an exemplary embodiment of the present disclosure;

FIG. 3B is a schematic diagram of the structure of an X-ray imaging system according to an exemplary embodiment of the present disclosure;

FIG. 4 is a schematic diagram of driving timing of a detection substrate according to an exemplary embodiment of the present disclosure;

FIG. 5 is a schematic diagram of the structure of a detection unit according to an exemplary embodiment of the present disclosure;

FIG. 6 is a schematic diagram of control signals of a gate driving unit and a data reading unit according to an exemplary embodiment of the present disclosure;

FIG. 7 is a schematic diagram of driving timing of a gate driving unit according to an exemplary embodiment of the present disclosure;

FIG. 8 is a schematic diagram of the structure of another X-ray imaging system according to an exemplary embodiment of the present disclosure;

FIGS. 9A to 9D are schematic diagrams of flows of four pre-correction methods for detection substrates according to exemplary embodiments of the present disclosure;

FIG. 10 is a schematic diagram of the structure of yet another X-ray imaging system according to an exemplary embodiment of the present disclosure;

FIG. 11 is a schematic diagram of a flow of an image correction method for a detection substrate according to an exemplary embodiment of the present disclosure;

FIG. 12 is a schematic diagram of the structure of an image correction apparatus for a detection substrate according to an exemplary embodiment of the present disclosure.

### DETAILED DESCRIPTION

To make objectives, technical solutions, and advantages of the present disclosure clearer, the embodiments of the present disclosure will be described in detail below with reference to the accompanying drawings. It is to be noted that the embodiments and features in the embodiments of the present disclosure may be randomly combined with each other if there is no conflict.

Unless otherwise defined, technical terms or scientific terms used in the embodiments of the present disclosure should have usual meanings understood by those of ordinary skills in the art to which the present disclosure belongs. “First”, “second”, and similar terms used in the embodiments of the present disclosure do not represent any order, quantity, or importance, but are only used for distinguishing different components. “Include”, “contain”, or a similar word means that an element or article appearing before the word covers an element or article and equivalent thereof listed after the word, and other elements or articles are not excluded.

As a core component of an X-ray imaging system, a Flat Panel Detector (FPD) is responsible for converting X-rays into electrical signals and recording images, which can be displayed in real time on a display or stored for subsequent reading. In general, an FPD includes a scintillator, a detector, a controller, a signal processing module, and a communication module. The scintillator absorbs X-rays to convert them into visible lights. The detector is composed of an array of detection units, with each detection unit being composed of a Photodiode and a Thin Film Transistor (TFT) switch, and converts visible lights generated by the scintillator into electrical signals under driving of the controller. Electrical signals are amplified by the signal processing module, converted into digital signals by an analog-to-digital converter, and are compensated to form images.

In practical applications, an X-ray sensor is usually built into the FPD, and the X-ray sensor can automatically detect whether X-rays are emitted. If X-rays are emitted, a signal is sent to the FPD for the FPD to be ready for exposure. This is the Automatic Exposure Detection (AED) technology. In an AED mode, if no X-rays are emitted, the FPD will be in a self-emptying state all the time. When an X-ray source emits X-rays, the X-ray sensor inside the FPD can immediately detect the X-rays and send an exposure request signal to the FPD, so that the FPD stops emptying itself to receive exposure. After the exposure is completed, the FPD collects electrical signals which are proportional to the X-ray irradiation intensity, and which form images after signal processing.

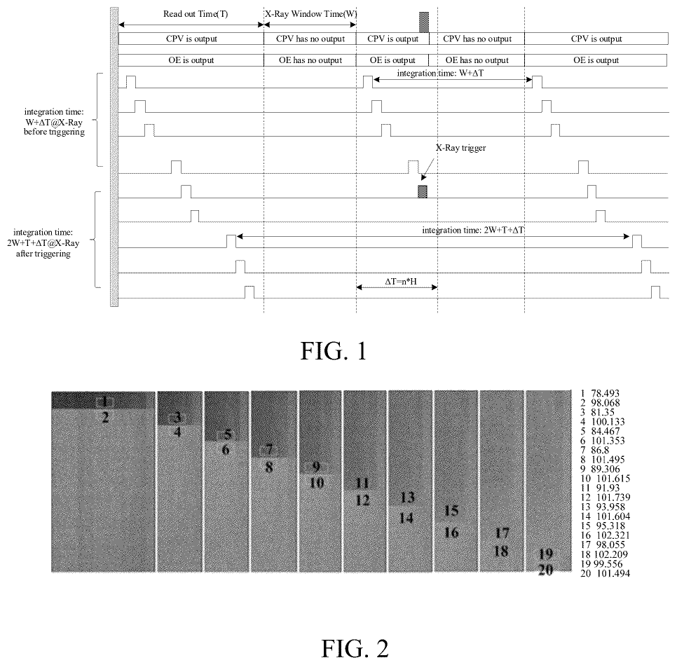

The FPD usually empties the charges in the pixels row by row by itself and receives exposure at a certain refresh rate. However, when the current FPD receives an exposure request signal, it immediately stops emptying itself to receive exposure. As shown in FIG. 1, taking an FPD with N rows of detection units (correspondingly, the image generated by the FPD has N rows of pixels) as an example, the FPD has been refreshed to a certain frame, and has emptied itself to an n-th (1<n<N) row under a self-emptying state. At this time, if an exposure request signal is received, the FPD will stop emptying itself immediately to start to receive exposure, that is, the FPD receives exposure from the (n+1)-th row to the N-th row, which will lead to a great difference between the gray scales displayed from the 1st row to the n-th row and the gray scales displayed from the (n+1)-th to the N-th row in a generated image, resulting a split-screen problem.

As can be seen from FIG. 1, the row integration time for rows before the currently interrupted row n is W+ΔT, and the row integration time for rows after the currently interrupted row is 2W+T+ΔT, where ΔT=n*H, H is scanning time for a single row. In the embodiment of the present disclosure, row integration time for a row of pixels refers to a time interval between the end of a previous row scanning operation and the start of the current row scanning operation by a detection unit corresponding to the row of pixels. The interruption is random, that is, the position n of the interrupted row is random, therefore AT is not a fixed value, and the gray scale difference which ultimately leads to the split upper screen and lower screen is also not a fixed value. FIG. 2 shows exemplary detection results of gray scale difference data in images interrupted at different positions in a flat panel detector. As shown in FIG. 2, when n=1, the gray scale difference between the split upper screen and lower screen is 98.068−78.493=19.575; when n=3, the gray scale difference between the split upper screen and lower screen is 100.133−81.35=18.783; . . . ; and when n=19, the gray scale difference between the split upper screen and lower screen is 101.494−99.556=1.938. In order to compensate for the problem that the gray scale difference between the split upper screen and lower screen is not fixed, some flat panel detectors collect gray scale differences for a series of different interrupted rows, calculate a linear fitting for the gray scale differences to form a linear equation. In a subsequent image correction process, a compensation value is calculated according to the interruption position of X-ray, and used to compensate for an image. However, lots of correction work is added in this process.

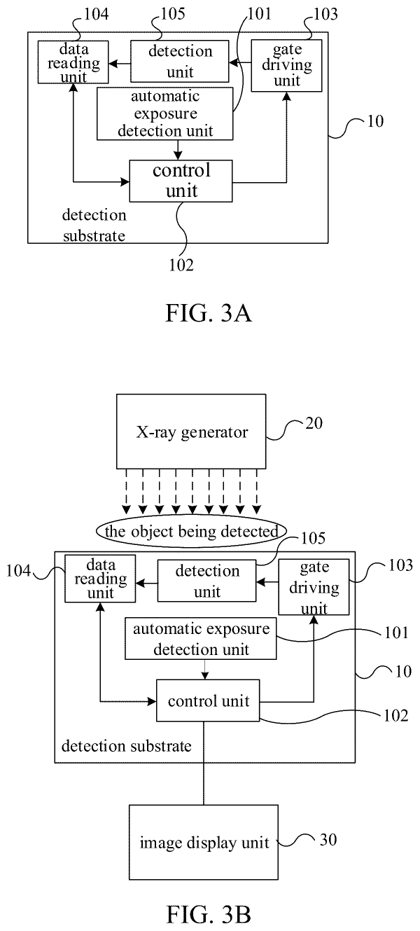

As shown in FIG. 3A, an embodiment of the present disclosure provides a detection substrate including an automatic exposure detection unit 101, a control unit 102, a gate driving unit 103, and a data reading unit 104. The detection substrate further includes a plurality of gate lines (not shown in FIG. 3A) and a plurality of data lines (not shown in FIG. 3A) arranged crosswise and a plurality of detection units 105 defined between the grid lines and the data lines, the plurality of detection units being arranged in an array, wherein:

the automatic exposure detection unit 101 is configured to transmit a first notification signal to the control unit 102 when detecting start of exposure;

the control unit 102 is configured to: output a first signal and a second signal to the gate driving unit 103 and output a data reading signal to the data reading unit 104 during a scanning time period; when the first notification signal is received during the scanning time period, output the first signal until the end of the current frame, and interrupt the output of the second signal; acquire data read by the data reading unit 104 during a first scanning time period after the exposure is completed, and generate an initial image according to the acquired data;

the gate driving unit 103 is configured to control an activation sequence of the plurality of detection units 105 according to the first signal and the second signal during the scanning time period, the first signal being used to determine the scanning time of a row in a frame, and the second signal being used to determine the position of the row of detection units which are turned on; and

the data reading unit 104 is configured to read data of the plurality of data lines according to a data reading signal during the scanning time period.

The detection substrate in the embodiment of the present disclosure adjusts driving timing of the control unit 102, and output the first signal until the end of the current frame, and interrupt the output of the second signal upon detection of start of exposure, so that the difference between the row integration time of rows before an interrupted row and the row integration time of rows after the interrupted row is fixed, that is, the difference between the row integration times is not affected by the position of the interrupted row, ensuring that the gray scale difference between the split upper screen and lower screen is a fixed value, and do not need much pre-correction work (for example, do not need to linearly fit a series of gray scale differences to form a linear equation), and a pre-stored fixed compensation value can be used to compensate the acquired images. Furthermore, since the pre-stored fixed compensation value is stored in advance before the detection substrate acquires the bright state images, the waiting time for forming an image is not increased when the detection substrate acquires bright state images.

In an embodiment of the present disclosure, a bright state image refers to an image acquired by a detection substrate in a scanning time period after an X-ray window time period, during an X-ray window time period, the detection substrate receives X-ray exposure. On the contrary, a dark state image refers to an image acquired by a detection substrate in a scanning time period after an X-ray window time period, during the X-ray window time period the detection substrate does not receive X-ray exposure.

In some exemplary implementations, the first signal may be a Clock Pulse Vertical (CPV) signal (also referred to as a gate row turning-on signal), and the second signal may be a gate Output Enable (OE) signal. However, the embodiments of the present disclosure are not limited thereto.

In some exemplary implementations, in the generated initial image, the row integration time of each row of pixels from the first row to the interrupted row is t1, the row integration time of each row of pixels from the interrupted row to the last row is t2, and the difference between t1 and t2 is a fixed value. An interrupted row is the row of detection units which are turned on when the second signal is interrupted, and the row integration time of a row of pixels is a time interval between end of a previous row scanning operation and start of the current row scanning operation by the detection units corresponding to the row of pixels.

In the embodiment of the present disclosure, the row integration time of the pixels in the interrupted row may be t1 or t2, depending on whether the scanning of the interrupted row has been completed when the output of the second signal is interrupted. In practice applications, the row integration time of the pixels in the interrupted row may be fixed to t1 or t2.

In some exemplary implementations, when the gate driving unit scans row by row from the first row to the last row, t1 is W+T, and t2 is 2W+2T;

and when the gate driving unit scans row by row from the last row to the first row, t2 is W+T, and t1 is 2W+2T, where T is scanning time of a row in a frame, and W is a preset exposure window width.

In some exemplary implementations, the control unit 102 is further configured to:

detect a position of an interrupted row when the output of the second signal is interrupted, calculate time for the current X-ray exposure according to the detected position of the interrupted row, wherein the time for the current X-ray exposure=T−ΔT+W, with T being a scanning time of a row in a frame, AT being a scanning time of a row before the current frame is interrupted, and W being a preset exposure window width, and send the calculated time for the current X-ray exposure to the X-ray generator so that the X-ray generator stops emitting X-rays according to the time for the current X-ray exposure.

In the embodiment of the present disclosure, the preset exposure window width W may be a time interval between end of a row scanning operation of the last row in a previous frame and start of a row scanning operation of the first row in a next frame. For example, the preset exposure window width W may be 1 second, however, the embodiment of the present disclosure is not limited thereto.

For example, the detection substrate is scanned row by row from the first row to the N-th row, therefore when the output of the second signal is interrupted, the control unit 102 calculates AT to be the scanning time of a row from the first row to the interrupted row. In another example, the detection substrate is scanned row by row from the N-th row to the first row, therefore when the output of the second signal is interrupted, the control unit 102 calculates ΔT to be the scanning time of a row from the interrupted row to the first row.

As shown in FIG. 3B, an embodiment of the present disclosure further provides an X-ray imaging system including an X-ray generator 20 and a detection substrate 10 as described in any embodiment of the present disclosure, wherein: the X-ray generator 20 is configured to emit X-rays.

In some exemplary implementations, the X-ray generator 20 is further configured to stop emitting X-rays according to a received time for the current X-ray exposure.

Still for example, the detection substrate is scanned row by row from the first row to the N-th row, therefore in a generated initial image, the row integration time of each row of pixels from the first row to the interrupted row is W+T, and the row integration time of each row of pixels from the interrupted row to the last row is 2W+2T, with the row integration time of a row of pixels being a time interval between end of a previous row scanning operation and start of the current row scanning operation by the detection units 105 corresponding to the row of pixels. In another example, the detection substrate is scanned row by row from the N-th row to the first row, therefore in the generated initial image, the row integration time of each row of pixels from the interrupted row to the last row is W+T, and the row integration time of each row of pixels from the first row to the interrupted row is 2W+2T.

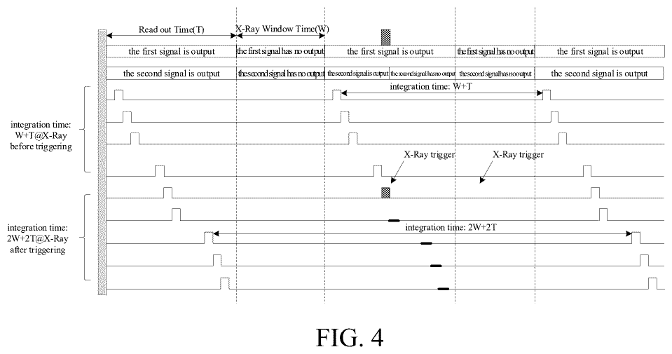

FIG. 4 is a schematic diagram of exemplary driving timing in the automatic exposure detection mode of the present disclosure. As shown in FIG. 4, still for example, the detection substrate is scanned row by row from the first row to the N-th row, and the whole driving process includes scanning time periods (read out time periods) T and X-Ray window time periods W that are periodically arranged. The X-Ray imaging system of the present disclosure is scanned row by row to empty, reset data lines and refresh the data lines with NULLs during a scanning time period (that is, in the embodiment of the present disclosure, data of the plurality of data lines is read out by the data reading unit 104, such that there is no data on the data lines, i.e., the data lines are emptied and reset, and since no X-ray exposure are received at this time, the data lines are considered to be refreshed with NULLs) until an X-ray signal is received. Once the automatic exposure detection unit 101 detects a triggering X-Ray signal, the control unit 102 immediately stops current acts of resetting and refreshing with NULLs. At this moment, the second signal (for example, it may be a gate output enable signal) used for controlling a gate signal to output a high level has no output, so that all gate signals of the subsequent rows output low levels, but the first signal (for example, it may be a clock pulse vertical signal) continues to be output until the end of the current frame, functioning as a timer. After the whole scanning time period T has ended, X-ray exposure is received in an X-ray window time period W, and after the X-ray window time period has ended, the detection substrate reads out an exposed bright state image row by row from the first row to the last row to acquire a whole image. As shown in FIG. 4, the row integration time for a row before the current interrupted row is W+T, and the row integration for a row after the current interrupted row is 2W+2T, therefor influence of the position of the interrupted row to the AT is eliminated, ensuring that the gray scale difference between the split upper screen and lower screen is a fixed value.

In practice tests, due to errors in the measuring instruments and for other reasons, there may be fluctuations in the gray scale differences between the split upper screen and lower screen in the X-ray images acquired in multiple times. However, the amplitudes of these fluctuations would not exceed 5% of the average of the gray scales in a whole image. Accordingly, there also may be fluctuations in differences between the row integration times before the current interrupted rows and the row integration times after the current interrupted rows. However, the amplitudes of these fluctuations would not exceed 5% of the average of multiple differences between row integration times.

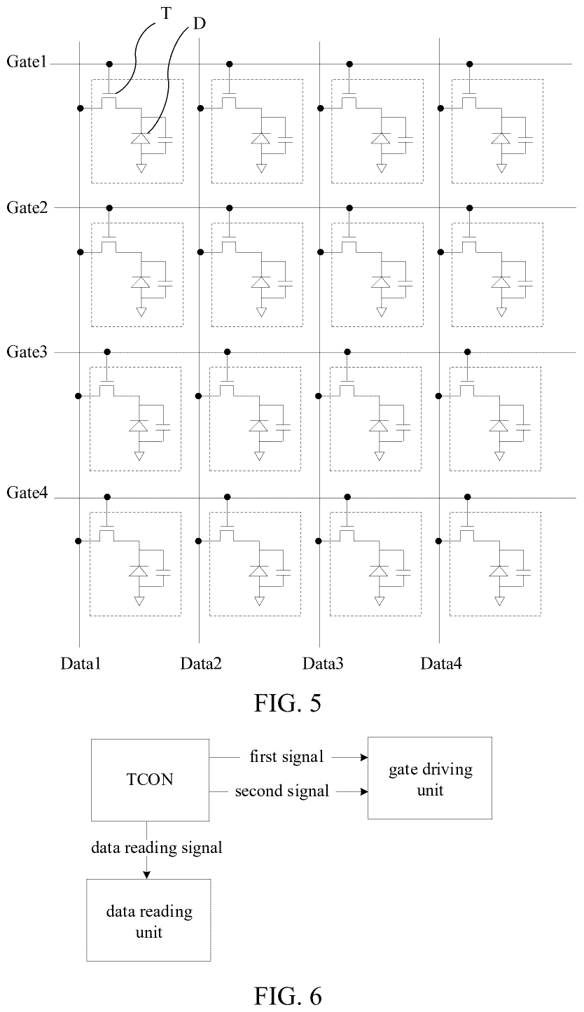

As shown in FIG. 5, the detection substrate includes a plurality of gate lines and a plurality of data lines arranged crosswise, and detection units 105 defined by the gate lines and the data lines, each detection unit 105 may include a photodiode D and a thin film transistor (TFT) T coupled with the photodiode D. Further, a thin film transistor T is also connected with a gate line and a data line. In operation, the X-rays transmitting through the human body are attenuated and converted into visible lights by a scintillator located on the surface of the detection substrate, and the photodiode D converts the visible lights into electric signals to form stored charges on the capacitor of the photodiode D. The gate scanning signals transmitted through the gate lines drives each row of detection units 105 to turn on sequentially to read out the stored charges in each row of detection units 105 through the data lines connected with the detection units 105, thereby forming X-ray digital images according to the stored charges. In the embodiment of the present disclosure, the photodiode D may be a PIN-type photodiode. A PIN-type photodiode is a widely used semiconductor photodetector which forms a PIN structure by adding an I region close to the intrinsic material between a P-type region and an N-type region with high doping concentrations.

In some exemplary implementations, as shown in FIG. 6, the control unit 102 includes a timing controller (TCON) configured to generate a plurality of control signals, such as a first signal, a second signal, a data reading signal, and the like. The timing controller inputs the generated first signal and the second signal to the gate driving unit 103, and inputs the generated data reading signal to the data reading unit 104. The gate driving unit 103 drives the plurality of gate lines to work in accordance with the first signal, the second signal, etc, and the data reading unit 104 reads out data on the data lines in accordance with the data reading signal, etc.

In the embodiment of the present disclosure, the gate driving unit 103 may be a gate driving circuit including a plurality of cascaded GOA (Gate on Array) circuit units, and may also be a gate driver, or the like, and the embodiment of the present disclosure is not limited thereto.

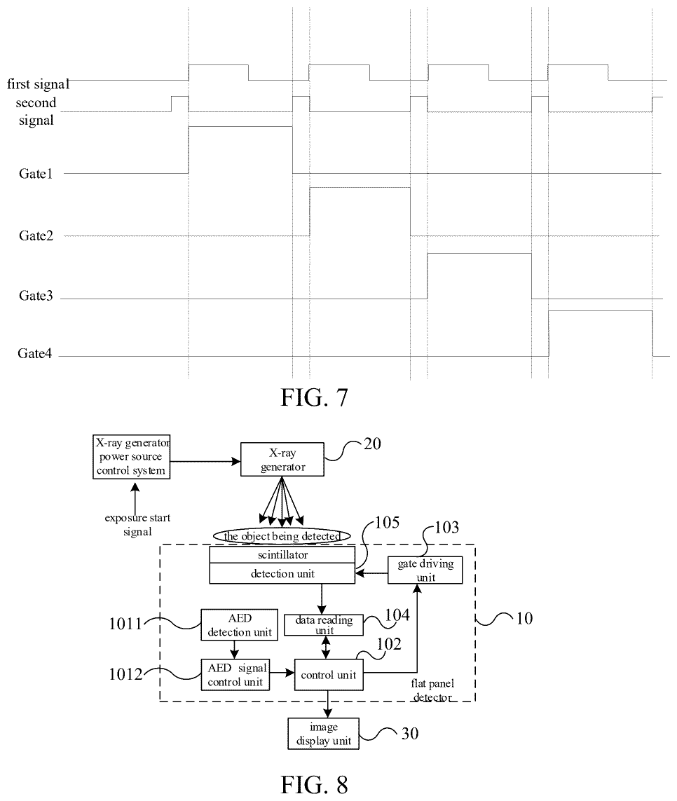

FIG. 7 is a schematic diagram of exemplary driving timing of a gate driving unit 103. As shown in FIG. 7, the falling edge of the i-th second signal corresponds to the rising edge of the i-th gate driving signal, and the rising edge of the (i+1)-th second signal corresponds to the falling edge of the (i−1)-th gate driving signal, so that the gate driving signals of each row of gate lines are output sequentially to sequentially turn on each row of detection units 105 to read data from the data lines.

In some exemplary implementations, as shown in FIG. 8, the automatic exposure detection unit 101 includes an AED detection unit 1011 and an AED signal control unit 1012, wherein:

the AED detection unit 1011 is configured to detect start of exposure, and to notify the AED signal control unit 1012 upon detecting start of exposure;

the AED signal control unit 1012 is configured to transmit a first notification signal to the control unit 102 upon receiving a notification from the AED detection unit 1011.

In some exemplary implementations, as shown in FIG. 8, the X-ray imaging system further includes an image display unit 30 which is configured to receive and display images output by the control unit 102.

In some exemplary implementations, the control unit 102 is further configured to:

obtain a position of the current interrupted row and a compensation value; use the compensation value to compensate for the gray scales of the pixels before the interrupted row (that is, pixels from the first row to the interrupted row) or after the interrupted row (that is, pixels from the interrupted row to the last row) in the initial image to obtain a corrected image.

Still for example, the detection substrate is scanned row by row from the first row to the N-th row, and using the compensation value to compensate for the gray scales of the pixels before the interrupted row in the initial image refers to using the compensation value to compensate for the gray scales of the pixels from the first row to the interrupted row to obtain a corrected image. For example, the pre-stored compensation value is 10, and 10 is added to the gray scale of each pixel from the first row to the interrupted row to obtain a corrected image.

And using the compensation value to compensate for the gray scales of the pixels after the interrupted row in the initial image refers to using the compensation value to compensate for the gray scales of the pixels from the interrupted row to the last row to obtain a corrected image. For example, the pre-stored compensation value is 10, and the gray level of each pixel from the interrupted row to the last row is subtracted by 10 to obtain a corrected image.

In some exemplary implementations, the compensation value is predetermined by a pre-correction method and stored in a storage device of the detection substrate.

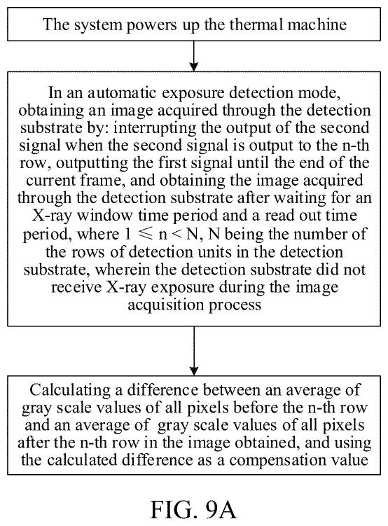

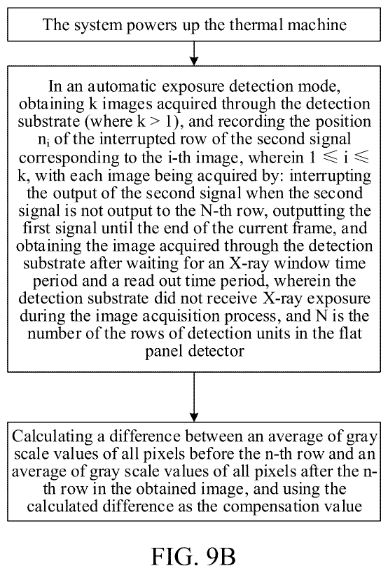

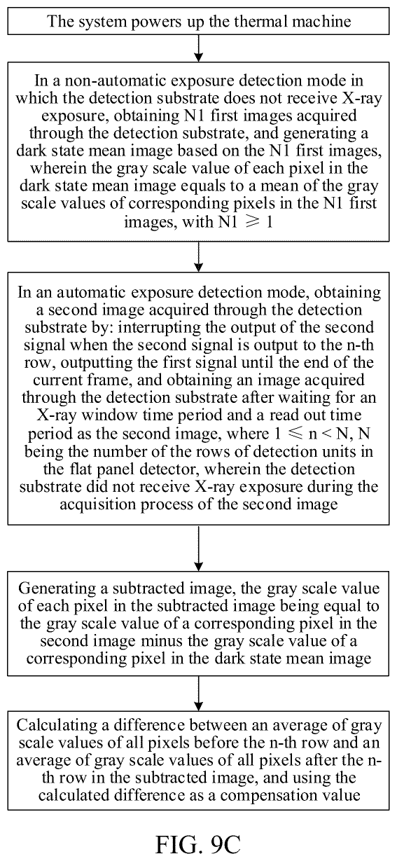

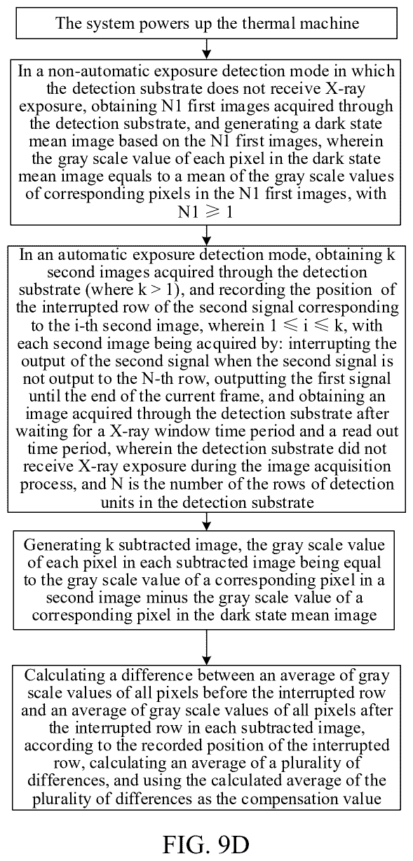

In some exemplary implementations, as shown in FIG. 9A, the compensation value may be predetermined by the following pre-correction method and stored in the storage device of the detection substrate:

in an automatic exposure detection mode, obtaining an image acquired through the detection substrate by: interrupting the output of the second signal when the second signal is output to the n-th row, outputting the first signal until the end of the current frame, and obtaining the image acquired through the detection substrate after waiting for an X-ray window time period and a scanning time period, where 1≤n<N, N being the quantity of the rows of detection units 105 in the detection substrate, wherein the detection substrate does not receive X-ray exposure during the image acquisition process;

calculating a difference between an average of gray scale values of all pixels before the n-th row and an average of gray scale values of all pixels after the n-th row in the image obtained, and using the calculated difference as a compensation value.

In the embodiment of the present disclosure, the compensation value is generated by a pre-correction method, and is stored in the storage device of the detection substrate in advance before a bright state image is acquired, so that when generating a bright state image, the waiting time for imaging is not increased, since the compensation value is easy and convenient for use.

In some other exemplary implementations, as shown in FIG. 9B, the compensation value may be predetermined by the following pre-correction method and stored in the storage device of the detection substrate:

in an automatic exposure detection mode, obtaining k images acquired through the detection substrate (where k>1), and recording the position nᵢ of the interrupted row of the second signal corresponding to the i-th image, wherein 1≤i≤k, with each image being acquired by: interrupting the output of the second signal when the second signal is not output to the N-th row, outputting the first signal until the end of the current frame, and obtaining the image acquired through the detection substrate after waiting for a X-ray window time period and a scanning time period, wherein the detection substrate does not receive X-ray exposure during the image acquisition process, and N is the quantity of the rows of detection units 105 in the detection substrate;

calculating a difference between an average of gray scale values of all pixels before the interrupted row and an average of gray scale values of all pixels after the interrupted row in each image, according to the recorded position of the interrupted row, calculating an average of a plurality of differences, and using the calculated average of the plurality of differences as the compensation value.

In the embodiment of the present disclosure, by obtaining k images acquired through the detection substrate during a pre-correction process, and calculating the average of a plurality of differences of gray scale values, the generated compensation value can be more reliable and accurate, and thus the imaging quality can be improved.

In some exemplary implementations, n₁ to nₖ constitute an arithmetic sequence.

By way of example, the detection substrate has 500 rows of detection units 105 in total, and n₁ to nₖ can be set to 49, 99, 149, 199, . . . , 449, 499 sequentially. At this time, the positions of the interrupted rows corresponding to the k images acquired through the detection substrate are 49, 99, 149, 199, . . . , 449, 499, respectively, so that k differences of gray scale values can be obtained to better reflect the differences between different interruption positions, and improve the final compensation effect.

Still in some other exemplary implementations, as shown in FIG. 9C, the compensation value may be predetermined by the following pre-correction method and stored in the storage device of the detection substrate:

in a non-automatic exposure detection mode in which the detection substrate does not receive X-ray exposure, obtaining N1 first images acquired through the detection substrate, and generating a dark state mean image according to the N1 first images, wherein the gray scale value of each pixel in the dark state mean image equals to a mean of the gray scale values of corresponding pixels in the N1 first images, with N1≥1;

in an automatic exposure detection mode, obtaining a second image acquired through the detection substrate by: interrupting the output of the second signal when the second signal is output to the n-th row, outputting the first signal until the end of the current frame, and obtaining an image acquired through the detection substrate after waiting for an X-ray window time period and a scanning time period as the second image, where 1≤n<N, N being the quantity of the rows of detection units 105 in the detection substrate, wherein the detection substrate does not receive X-ray exposure during the acquisition process of the second image;

generating a subtracted image, the gray scale value of each pixel in the subtracted image being equal to the gray scale value of a each pixel in the second image minus the gray scale value of a each pixel in the dark state mean image;

calculating a difference between an average of gray scale values of all pixels before the n-th row and an average of gray scale values of all pixels after the n-th row in the subtracted image, and using the calculated difference as a compensation value.

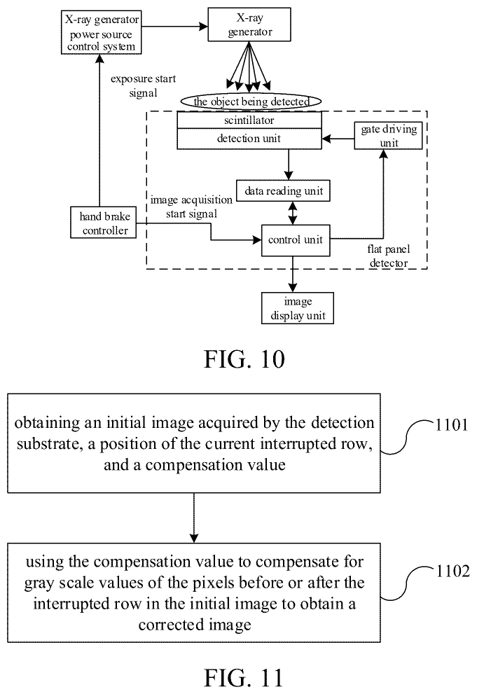

As shown in FIG. 10, in the non-automatic exposure detection mode, a hand brake controller is needed to trigger an exposure start signal and an image acquisition start signal. The exposure start signal is used to trigger the X-ray generator to start the exposure process, and the image acquisition start signal is used to trigger the detection substrate to start to acquire image. Therefore, the exposure will not start in a scanning time period, that is, the problem of split upper screen and lower screen will not occur.

Still in some other exemplary implementations, as shown in FIG. 9D, the compensation value is also predetermined by the following pre-correction method and stored in the storage device of the detection substrate:

in a non-automatic exposure detection mode in which the detection substrate does not receive X-ray exposure, obtaining N1 first images acquired through the detection substrate, and generating a dark state mean image according to the N1 first images, wherein the gray scale value of each pixel in the dark state mean image equals to a mean of the gray scale values of corresponding pixels in the N1 first images, with N1≥1;

in an automatic exposure detection mode, obtaining k second images acquired through the detection substrate (where k>1), and recording the position nᵢ of the interrupted row of the second signal corresponding to the i-th second image, wherein 1≤i≤k, with each second image being acquired by: interrupting the output of the second signal when the second signal is not output to the N-th row, outputting the first signal until the end of the current frame, and obtaining the image acquired through the detection substrate after waiting for a X-ray window time period and a scanning time period, as the second image, wherein the detection substrate does not receive X-ray exposure during the acquisition process of the second image, and N is the quantity of the rows of detection units 105 in the detection substrate;

generating k subtracted image, the gray scale value of each pixel in each subtracted image being equal to the gray scale value of a each pixel in a second image minus the gray scale value of a corresponding pixel in the dark state mean image;

calculating a difference between an average of gray scale values of all pixels before the interrupted row and an average of gray scale values of all pixels after the interrupted row in each subtracted image, according to the recorded position of the interrupted row, calculating an average of a plurality of differences, and using the calculated average of the plurality of differences as the compensation value.

In the embodiment of the present disclosure, a fixed gray scale difference between the split upper screen and lower screen is obtained for using as a compensation value by correcting the image and adjusting the timing in advance. In a subsequent bright state image acquisition process, the same timing is used to obtain an initial bright state image, and a position of the interrupted row is recorded. Using the compensation value (i.e., the fixed gray scale difference that is calculated), the initial bright state image is corrected before it is output, obtaining a corrected bright state image with no split-screen problem. Since the gray scale value for correction has been determined and stored in advance before a bright state image is acquired, the waiting time for imaging will not be increased.

As shown in FIG. 11, an embodiment of the present disclosure further provides an image correction method for a detection substrate, the method comprising:

Step 1101: obtaining an initial image, a position of the current interrupted row and a compensation value acquired by the detection substrate, wherein the position of the interrupted row is a position of the row being scanned when start of exposure is detected and the output of the second signal is interrupted;

Step 1102: using the compensation value to compensate for gray scale values of the pixels before or after the interrupted row in the initial image to obtain a corrected image.

In some exemplary implementation, in step 1102, gray scale values of the pixels from the first row to the interrupted row may be compensated by using the compensation value to obtain a corrected image. For example, the pre-stored compensation value is 10, and 10 is added to the gray scale of each pixel from the first row to the interrupted row to obtain a corrected image.

In some exemplary implementations, in step 1102, gray scale values of the pixels from the interrupted row to the last row may be compensated by using the compensation value to obtain a corrected image. For example, the pre-stored compensation value is 10, and the gray scale of each pixel from the interrupted row to the last row is subtracted by 10 to obtain a corrected image.

In some exemplary implementations, the compensation value is predetermined by the following pre-correction method and stored in the storage device of the detection substrate:

in an automatic exposure detection mode, obtaining an image acquired through the detection substrate by: interrupting the output of the second signal when the second signal is output to the n-th row, outputting the first signal until the end of the current frame, and obtaining the image acquired through the detection substrate after waiting for an X-ray window time period and a scanning time period, where 1≤n<N, N being the quantity of the rows of detection units in the detection substrate, wherein the detection substrate does not receive X-ray exposure during the image acquisition process;

calculating a difference between an average of gray scale values of all pixels before the n-th row and an average of gray scale values of all pixels after the n-th row in the image obtained, and using the calculated difference as a compensation value.

In some other exemplary implementations, the compensation value is predetermined by the following pre-correction method and stored in the storage device of the detection substrate:

in an automatic exposure detection mode, obtaining k images acquired through the detection substrate (where k>1), and recording the position nᵢ of the interrupted row of the second signal corresponding to the i-th image, wherein 1≤i≤k, with each image being acquired by: interrupting the output of the second signal when the second signal is not output to the N-th row, outputting the first signal until the end of the current frame, and obtaining the image acquired through the detection substrate after waiting for a X-ray window time period and a scanning time period, wherein the detection substrate does not receive X-ray exposure during the image acquisition process, and N is the quantity of the rows of detection units in the detection substrate;

calculating a difference between an average of gray scale values of all pixels before the interrupted row and an average of gray scale values of all pixels after the interrupted row in each image, according to the recorded position of the interrupted row, calculating an average of a plurality of differences, and using the calculated average of the plurality of differences as the compensation value.

In some exemplary implementations, n₁ to nₖ constitute an arithmetic sequence.

In yet some other exemplary implementations, the compensation value is predetermined by the following pre-correction method and stored in the storage device of the detection substrate:

in a non-automatic exposure detection mode in which the detection substrate does not receive X-ray exposure, obtaining N1 first images acquired through the detection substrate, and generating a dark state mean image according to the N1 first images, wherein the gray scale value of each pixel in the dark state mean image equals to a mean of the gray scale values of corresponding pixels in the N1 first images, with N1≥1;

in an automatic exposure detection mode, obtaining a second image acquired through the detection substrate by: interrupting the output of the second signal when the second signal is output to the n-th row, outputting the first signal until the end of the current frame, and obtaining an image acquired through the detection substrate after waiting for an X-ray window time period and a scanning time period as the second image, where 1≤n<N, N being the quantity of the rows of detection units in the detection substrate, wherein the detection substrate does not receive X-ray exposure during the acquisition process of the second image;

generating a subtracted image, the gray scale value of each pixel in the subtracted image being equal to the gray scale value of a each pixel in the second image minus the gray scale value of a each pixel in the dark state mean image;

calculating a difference between an average of gray scale values of all pixels before the n-th row and an average of gray scale values of all pixels after the n-th row in the subtracted image, and using the calculated difference as a compensation value.

In yet some other exemplary implementations, the compensation value is predetermined by the following pre-correction method and stored in the storage device of the detection substrate:

in a non-automatic exposure detection mode in which the detection substrate does not receive X-ray exposure, obtaining N1 first images acquired through the detection substrate, and generating a dark state mean image according to the N1 first images, wherein the gray scale value of each pixel in the dark state mean image equals to a mean of the gray scale values of corresponding pixels in the N1 first images, with N1≥1;

in an automatic exposure detection mode, obtaining k second images acquired through the detection substrate (where k>1), and recording the position nᵢ of the interrupted row of the second signal corresponding to the i-th second image, wherein 1≤i≤k, with each second image being acquired by: interrupting the output of the second signal when the second signal is not output to the N-th row, outputting the first signal until the end of the current frame, and obtaining the image acquired through the detection substrate after waiting for a X-ray window time period and a scanning time period, as the second image, wherein the detection substrate does not receive X-ray exposure during the acquisition process of the second image, and N is the quantity of the rows of detection units in the detection substrate;

generating k subtracted image, the gray scale value of each pixel in each subtracted image being equal to the gray scale value of a each pixel in a second image minus the gray scale value of a corresponding pixel in the dark state mean image;

calculating a difference between an average of gray scale values of all pixels before the interrupted row and an average of gray scale values of all pixels after the interrupted row in each subtracted image, according to the recorded position of the interrupted row, calculating an average of a plurality of differences, and using the calculated average of the plurality of differences as the compensation value.

An embodiment of the present disclosure further provides an image correction apparatus for a detection substrate, the image correction apparatus includes a memory and a processor coupled to the memory for storing instructions, the processor is configured to perform the steps of the image correction method for the detection substrate described in any embodiment of the present disclosure.

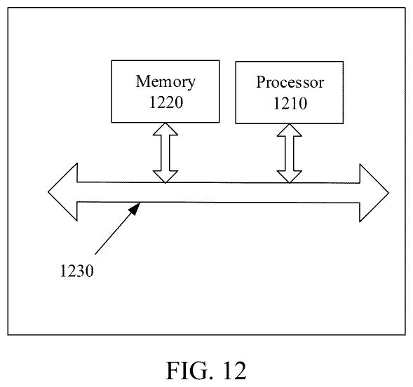

As shown in FIG. 12, in one example, the image correction apparatus for the detection substrate may include a processor 1210, a memory 1220, and a bus system 1230, wherein the processor 1210 and the memory 1220 are connected via the bus system 1230, the memory 1220 is used to store instructions, and the processor 1210 is used to execute the instructions stored in the memory 1220 to obtain an initial image acquired by the detection substrate, a position of the current interrupted row, and a compensation value, the position of the interrupted row being the position of a scanned row where start of exposure is detected and the output of the second signal is interrupted. The compensation value is used to compensate for gray scale values of the pixels before or after the interrupted row in the initial image to obtain a corrected image.

It should be understood that the processor 1210 may be a Central Processing Unit (CPU), or the processor 1210 may be another general-purpose processor, a Digital Signal Processor (DSP), an Application Specific Integrated Circuit (ASIC), a Field Programmable Gate Array (FPGA) or another programmable logic device, a discrete gate or a transistor logic device, a discrete hardware component, etc. The general-purpose processor may be a microprocessor, or the processor may be any conventional processor, etc.

The memory 1220 may include a read only memory and a random access memory, and provides instructions and data to the processor 1210. A portion of the memory 1220 may further include a non-volatile random access memory. For example, the memory 1220 may store information of a device type.

The bus system 1230 may also include a power bus, a control bus, a status signal bus, or the like in addition to a data bus. However, for clarity of illustration, various buses are all denoted as the bus system 1230 in FIG. 12.

In an implementation process, processing performed by a processing device may be completed through an integrated logic circuit of hardware in the processor 1210 or instructions in a form of software. That is, acts of the method in the embodiments of the present disclosure may be embodied as executed and completed by a hardware processor, or executed and completed by a combination of hardware in the processor and a software module. The software module may be located in a storage medium such as a random access memory, a flash memory, a read only memory, a programmable read only memory or an electrically erasable programmable memory, a register. The storage medium is located in the memory 1220, and the processor 1210 reads information in the memory 1220 and completes the acts of the above method in combination with its hardware. In order to avoid repetition, detailed description is not provided here.

An embodiment of the present disclosure further provides a computer-readable storage medium storing a computer program thereon, the program, when executed by the processor, implements the image correction method for the detection substrate described in any embodiment of the present disclosure. The image correction method for the detection substrate driven by executing executable instructions is basically the same as the image correction method for the detection substrate provided in the above-described embodiment of the present disclosure, and will not be repeatedly described here.

In some possible implementations, the various aspects of the image correction method for a detection substrate provided herein may also be implemented in the form of a program product, which includes a program code. When the program product is run on a computer device, the program code is used to enable the computer device to perform the acts in the image correction method for the detection substrate described above in this specification according to various exemplary implementations of the present application, for example, the computer device may perform the image correction method for the detection substrate described in embodiments of the present application.

For the program product, any combination of one or more readable media may be used. A readable medium may be a readable signal medium or a readable storage medium. The readable storage medium may be, for example, but is not limited to, an electrical, magnetic, optical, electromagnetic, infrared, or semiconductor system, apparatus, or device, or a combination of any of the above. More specific examples (non-exhaustive list) of the readable storage medium include: an electrical connection with one or more wires, a portable disk, a hard disk, a Random Access Memory (RAM), a Read Only Memory (ROM), an Erasable Programmable Read Only Memory (EPROM or flash memory), an optical fiber, a portable Compact Disk Read Only Memory (CD-ROM), an optical storage device, a magnetic storage device, or any suitable combination of the above.

Those of ordinary skills in the art may understand that all or some of acts in the methods disclosed above, systems, functional modules or units in apparatuses may be implemented as software, firmware, hardware, and an appropriate combination thereof. In a hardware implementation mode, division of the function modules/units mentioned in the above description is not always corresponding to division of physical components. For example, a physical component may have multiple functions, or a function or an act may be executed by several physical components in cooperation. Some components or all components may be implemented as software executed by a processor such as a digital signal processor or a microprocessor, or implemented as hardware, or implemented as an integrated circuit such as an application specific integrated circuit. Such software may be distributed on a computer readable medium, and the computer readable medium may include a computer storage medium (or a non-transitory medium) and a communication medium (or a transitory medium). As known to those of ordinary skills in the art, a term computer storage medium includes volatile and nonvolatile, and removable and irremovable media implemented in any method or technology for storing information (for example, a computer readable instruction, a data structure, a program module, or other data). The computer storage medium includes, but is not limited to, a RAM, a ROM, an Electrically Erasable Programmable Read Only Memory (EEPROM), a flash memory or another memory technology, a Compact Disc Read Only Memory (CD-ROM), a Digital Versatile Disk (DVD) or another optical disk storage, a magnetic cartridge, a magnetic tape, magnetic disk storage or another magnetic storage apparatus, or any other medium that may be configured to store desired information and may be accessed by a computer. In addition, it is known to those of ordinary skill in the art that the communication medium usually includes a computer readable instruction, a data structure, a program module, or other data in a modulated data signal of, such as, a carrier wave or another transmission mechanism, and may include any information delivery medium.

Although implementations disclosed in the present disclosure are described as above, the described contents are only implementations which are used for facilitating understanding of the present disclosure, but are not intended to limit the present invention. Any skilled person in the art to which the present disclosure pertains may make any modification and variation in a form and details of implementation without departing from the spirit and scope of the present disclosure. However, the patent protection scope of the present invention should be subject to the scope defined in the appended claims.

1. A detection substrate, comprising an automatic exposure detection unit, a control unit, a gate driving unit, and a data reading unit, wherein the detection substrate further comprises a plurality of gate lines and a plurality of data lines arranged crosswise and a plurality of detection units defined between the gate lines and the data lines, the plurality of detection units being arranged in an array, wherein:
the automatic exposure detection unit is configured to transmit a first notification signal to the control unit when detecting a start of exposure;
the control unit is configured to output a first signal and a second signal to the gate driving unit and output a data reading signal to the data reading unit during a scanning time period;
when the first notification signal is received during the scanning time period, output the first signal until an end of a current frame, and interrupt an output of the second signal; acquire data read by the data reading unit during a first scanning time period after the exposure is completed, and generate an initial image according to the acquired data;
the gate driving unit is configured to control an activation sequence of the plurality of detection units according to the first signal and the second signal during the scanning time period, the first signal being used to determine a scanning time of a row in a frame, and the second signal being used to determine a position of a row of detection units which are turned on; and
the data reading unit is configured to read data of the plurality of data lines according to the data reading signal during the scanning time period.

2. The detection substrate according to claim 1, wherein in the generated initial image, row integration time of each row of pixels from a first row to an interrupted row is t1, row integration time of each row of pixels from the interrupted row to a last row is t2, and difference between t1 and t2 is a fixed value, and the interrupted row is a row of detection units which are turned on when the second signal is interrupted, and row integration time of a row of pixels is a time interval between an end of a previous row scanning operation and a start of a current row scanning operation by detection units corresponding to the row of pixels.

3. The detection substrate according to claim 2, wherein t1 is W+T and t2 is 2W+2T when the gate driving unit scans row by row from the first row to the last row; and
t2 is W+T, and t1 is 2W+2T when the gate driving unit scans row by row from the last row to the first row, where T is scanning time of a row in a frame, and W is a preset exposure window width.

4. The detection substrate according to claim 2, wherein the control unit is further configured to:
obtain a predetermined compensation value; and
use the compensation value to compensate for gray scale of pixels before or after the interrupted row in the initial image to obtain a corrected image.

5. The detection substrate according to claim 4, wherein the compensation value is predetermined by a pre-correction method and stored in a storage device of the detection substrate.

6. An X-ray imaging system comprising an X-ray generator and the detection substrate according to claim 1, wherein:
the X-ray generator is configured to emit X-rays, and stop emitting X-rays according to received time for current X-ray exposure;
the control unit is further configured to detect a position of an interrupted row when the output of the second signal is interrupted, calculate the time for the current X-ray exposure according to the detected position of the interrupted row, wherein the time for the current X-ray exposure=T−ΔT+W, wherein T is a scanning time of a row in a frame, ΔT is a scanning time of a row before the current frame is interrupted, and W is a preset exposure window width, and send the calculated time for the current X-ray exposure to the X-ray generator so that the X-ray generator stops emitting X-rays according to the time for the current X-ray exposure.

7. An image correction method for the detection substrate according to claim 1, comprising:
obtaining the initial image, a position of a current interrupted row and a compensation value acquired by the detection substrate, wherein the position of the interrupted row is a position of a row being scanned when a start of exposure is detected and the output of the second signal is interrupted;
using the compensation value to compensate for gray scale of pixels before or after the interrupted row in the initial image to obtain a corrected image.

8. The image correction method according to claim 7, wherein the compensation value is pre-determined by a following pre-correction method and stored in a storage device of the detection substrate:
in an automatic exposure detection mode, obtaining an image acquired through the detection substrate by: interrupting the output of the second signal when the second signal is output to a n-th row, outputting the first signal until the end of the current frame, and obtaining the image acquired through the detection substrate after waiting for an X-ray window time period and a scanning time period, where 1≤n<N, N being a quantity of the rows of detection units in the detection substrate, wherein the detection substrate does not receive X-ray exposure during an image acquisition process;
calculating a difference between an average of gray scale values of all pixels before the n-th row and an average of gray scale values of all pixels after the n-th row in the image obtained, and using the calculated difference as a compensation value.

9. The image correction method according to claim 7, wherein the compensation value is pre-determined by a following pre-correction method and stored in a storage device of the detection substrate:
in an automatic exposure detection mode, obtaining k images acquired through the detection substrate (where k>1), and recording a position nᵢ of the interrupted row of the second signal corresponding to an i-th image, wherein 1≤i≤k, with each image being acquired by: interrupting the output of the second signal when the second signal is not output to a N-th row, outputting the first signal until the end of the current frame, and obtaining the image acquired through the detection substrate after waiting for a X-ray window time period and a scanning time period, wherein the detection substrate does not receive X-ray exposure during an image acquisition process, and N is a quantity of the rows of detection units in the detection substrate;
calculating a difference between an average of gray scale values of all pixels before the interrupted row and an average of gray scale values of all pixels after the interrupted row in each image, according to the recorded position of the interrupted row, calculating an average of a plurality of differences, and using the calculated average of the plurality of differences as the compensation value.

10. The image correction method according to claim 9, wherein n₁ to nₖ constitute an arithmetic sequence.

11. The image correction method according to claim 7, wherein the compensation value is pre-determined by a following pre-correction method and stored in a storage device of the detection substrate:
in a non-automatic exposure detection mode in which the detection substrate does not receive X-ray exposure, obtaining N1 first images acquired through the detection substrate, and generating a dark state mean image according to the N1 first images, wherein a gray scale value of each pixel in the dark state mean image equals to a mean of gray scale values of corresponding pixels in the N1 first images, with N1≥1;
in an automatic exposure detection mode, obtaining a second image acquired through the detection substrate by: interrupting the output of the second signal when the second signal is output to a n-th row, outputting the first signal until the end of the current frame, and obtaining an image acquired through the detection substrate as the second image after waiting for an X-ray window time period and a scanning time period, where 1≤n<N, N being a quantity of the rows of detection units in the detection substrate, wherein the detection substrate does not receive X-ray exposure during an acquisition process of the second image;
generating a subtracted image, a gray scale value of each pixel in the subtracted image being equal to a gray scale value of a each pixel in the second image minus a gray scale value of a each pixel in the dark state mean image;
calculating a difference between an average of gray scale values of all pixels before the n-th row and an average of gray scale values of all pixels after the n-th row in the subtracted image, and using the calculated difference as the compensation value.

12. The image correction method according to claim 7, wherein, the compensation value is pre-determined by a following pre-correction method and stored in a storage device of the detection substrate:
in a non-automatic exposure detection mode in which the detection substrate does not receive X-ray exposure, obtaining N1 first images acquired through the detection substrate, and generating a dark state mean image according to the N1 first images, wherein a gray scale value of each pixel in the dark state mean image equals to a mean of gray scale values of corresponding pixels in the N1 first images, with N1≥1;
in an automatic exposure detection mode, obtaining k second images acquired through the detection substrate (where k>1), and recording a position n; of the interrupted row of the second signal corresponding to an i-th second image, wherein 1≤i≤k, with each second image being acquired by: interrupting the output of the second signal when the second signal is not output to a N-th row, outputting the first signal until the end of the current frame, and obtaining the image acquired through the detection substrate after waiting for a X-ray window time period and a scanning time period, as the second image, wherein the detection substrate does not receive X-ray exposure during an acquisition process of the second image, and N is a quantity of the rows of detection units in the detection substrate;
generating k subtracted image, a gray scale value of each pixel in each subtracted image being equal to a gray scale value of a each pixel in a second image minus a gray scale value of a corresponding pixel in the dark state mean image;
calculating a difference between an average of gray scale values of all pixels before the interrupted row and an average of gray scale values of all pixels after the interrupted row in each subtracted image, according to the recorded position of the interrupted row, calculating an average of a plurality of differences, and using the calculated average of the plurality of differences as the compensation value.

13. An image correction apparatus for a detection substrate, comprising a memory and a processor connected to the memory for storing instructions, wherein the processor is configured to perform acts of the image correction method for the detection substrate according to claim 7 based on the instructions stored in the memory.

14. A computer readable storage medium on which a computer program is stored, wherein when the program is executed by a processor, the image correction method for the detection substrate according to claim 7 is implemented.
